(19) United States
(12) Patent Application Publication
(10) Pub. No.: US 2025/0367402 A1
(43) Pub. Date: Dec. 4, 2025

(54) SYSTEMS AND METHODS FOR MANUFACTURING INFUSION CANNULAS

(71) Applicant: Tandem Diabetes Care, Inc., San Diego, CA (US)

(72) Inventors: Justin Coker, San Diego, CA (US); Kennie Hsu, San Diego, CA (US); Amrith Karunaratne, San Diego, CA (US)

(21) Appl. No.: 19/221,933

(22) Filed: May 29, 2025

Related U.S. Application Data
(60) Provisional application No. 63/652,954, filed on May 29, 2024.

(51) Int. Cl. A61M 25/00; A61M 5/142; A61M 5/158; B21C 23/08

(52) U.S. Cl. CPC A61M 25/0013; B21C 23/085; A61M 5/14248; A61M 5/158

(57) ABSTRACT

A system and method for manufacturing a plurality of cannulas configured to facilitate the delivery of medicament to a user having internal coils can include providing spacers along portions of a mandrel between the coils. Spacers prevent shifting of the coils both during assembly on the mandrel and during the extrusion process, which will ensure sufficient material extending beyond the coil for creation of the tapered distal end for the cannula. Use of spacers also provides a more uniform outer wall.

### PRIORITY CLAIM

The present application claims the benefit of U.S. Provisional Application No. 63/652,954 filed May 29, 2024, which is hereby incorporated by reference in its entirety.

### TECHNICAL FIELD

The present disclosure relates to manufacturing of infusion sets that facilitate injection of medicament into a body of a patient.

### BACKGROUND

There are a wide variety of medical treatments that include the administration of a therapeutic fluid in precise, known amounts at predetermined intervals. Devices and methods that are directed to the delivery of such fluids, which may be liquids or gases, are known in the art.

One category of such fluid delivery devices includes insulin injecting pumps developed for administering insulin to patients afflicted with Type 1 or Type 2 diabetes. Some insulin injecting pumps are configured as portable or ambulatory infusion devices that can provide continuous subcutaneous insulin injection and/or infusion therapy as an alternative to multiple daily injections of insulin via a syringe or an insulin pen. Such pumps can be worn or carried by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Some portable infusion pumps deliver medicament to patients through infusion sets that include tubing extending from the pump and a cannula with an associated needle positioned transcutaneously (i.e., through the patient's skin) at an infusion site to allow infusion of the medicament through a cannula and into the patient. Such pumps can be worn on the body or carried near the body (e.g., in the user's pocket) with the infusion site situated on the patient's body and connected with the pump via the tubing. Other pumps that are worn directly on the body can deliver medicament through a cannula that extends directly beneath the pump.

If a patient leaves the cannula injected at the infusion site at one location for too long a period of time, or uses the same infusion site repeatedly, unwanted side effects such as infection and the accumulation of fat and scar tissue can result. Therefore, patients are often instructed to rotate infusion sites to avoid or minimize side effects. Depending on the type of cannula used, the general physiological response of the patient with regard to insulin absorption, and other factors, the time needed between infusion site rotations can vary. Commonly, sites are rotated every 24-48 hours or every 48-72 hours and extended wear infusion sets that can be worn for a longer period of time, such as for example, 7 days, are also being developed.

Embodiments of various infusion sets are described in U.S. Patent Publication Nos. 2018/0280608, 2021/0402084, 2022/0226568 and 2023/0277765, each of which is hereby incorporated herein by reference in its entirety. Some of these infusion sets include a coil extending through the delivery cannula inserted into the user for delivery of medicament into the user's skin. Use of such a coil within the cannula can provide a number of benefits to the user including, but not limited to, flexibility and kink resistance, which increase the wear time of an infusion set. The coil can also provide a filtering function to capture and retain aggregate particles formed in the liquid medication. Such aggregate particles can induce or contribute to infusion site inflammatory and immune responses if delivered and can compromise the infusion site.

While cannulas with internal coils provide significant advantages, there are technical challenges and increased costs associated with the manufacturing process. For example, the coils must be manually inserted on a long mandrel, the coils must be manually spaced and aligned along the length of the mandrel, care must be taken so that the coils do not shift during the extrusion process, the mandrel must be removed from the extruded coils, and the extruded coils must be cut to size. These requirements, alone or in combination with one another, can be a challenge for large scale manufacturing.

### SUMMARY

A system and method for manufacturing a plurality of cannulas configured to facilitate the delivery of medicament to a user having internal coils can include providing spacers along portions of a mandrel between the coils. Spacers prevent shifting of the coils both during assembly on the mandrel and during the extrusion process, which will ensure sufficient material extending beyond the coil for creation of the tapered distal end for the cannula. Use of spacers also provides a more uniform outer wall.

In an embodiment, a method of manufacturing a plurality of cannulas configured to facilitate delivery of medicament to a user can including arranging a plurality of coils on a mandrel and providing a spacer on the mandrel between each of the coils to form an assembly having alternating coils and spacers along the mandrel. The assembly can be processed through an extrusion device to form an outer tube around the assembly. The processed assembly can be cut adjacent both ends of each coil to create a plurality of cannula sections. A cannula configured to facilitate delivery of medicament can be formed from each cannula section.

In an embodiment, a cannula configured to facilitate delivery of medicament to a user is formed by a process comprising arranging a plurality of coils on a mandrel and providing a spacer on the mandrel between each of the coils to form an assembly having alternating coils and spacers along the mandrel. The assembly can be processed through an extrusion device to form an outer tube around the assembly. The processed assembly can be cut adjacent both ends of each coil to create a plurality of cannula sections. A cannula configured to facilitate delivery of medicament can be formed from each cannula section.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict an ambulatory infusion pump with an infusion set according to an embodiment of the disclosure.

FIGS. 2A-2C depict an ambulatory infusion pump according to an embodiment of the disclosure.

FIG. 3 depicts an infusion set according to an embodiment of the disclosure.

FIGS. 4A-4C depict a cannula according to an embodiment of the disclosure.

FIGS. 5-5C depict an assembly used in creating a plurality of cannulas according to an embodiment of the disclosure.

FIGS. 6-6B depict an assembly used in creating a plurality of cannulas according to an embodiment of the disclosure.

FIG. 7 depicts a flowchart of steps for a method of manufacturing a cannula according to an embodiment of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

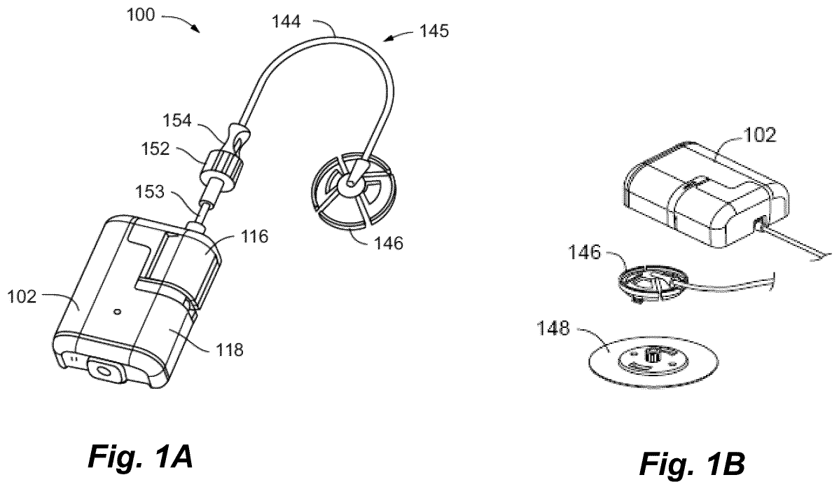

FIGS. 1A-1B depict an exemplary medical device that can be used with embodiments of the disclosure. In this embodiment, the medical device is configured as a pump 102, such as an infusion pump, that can include a pumping or delivery mechanism and a reservoir for delivering a medicament to a patient. In one embodiment, the medical device can be a portable pump configured to deliver insulin to a patient. Further details regarding such pump devices can be found in U.S. Pat. Nos. 8,287,495, 10,279,107 and 10,864,318, each of which is incorporated herein by reference in its entirety. In other embodiments, the medical device can be an infusion pump configured to deliver one or more additional or other medicaments to a patient.

As depicted in FIGS. 1A-1B, pump system 100 can include a pump 102 and an infusion set 145. In embodiments, pump system 100 can include a drive unit 118 and a cartridge 116 having a short length of tubing 153 and a connector 152 extending therefrom. Infusion set 145 can include tubing 144 extending between a connector 154 and a site connector 146. Connector 154 on infusion set 145 can be configured to couple to pump 102 at connector 152 of pump 102. As shown in FIG. 1B, site connector 146 can be configured to be attached to an infusion hub 148 at an infusion site on a user through which medicament from the pump is delivered to a patient through a cannula extending from the infusion hub 148 into the user's skin.

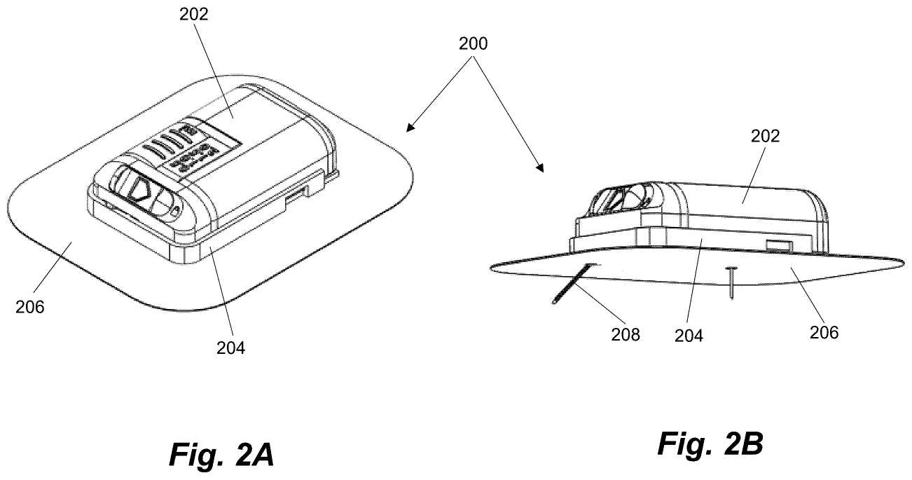

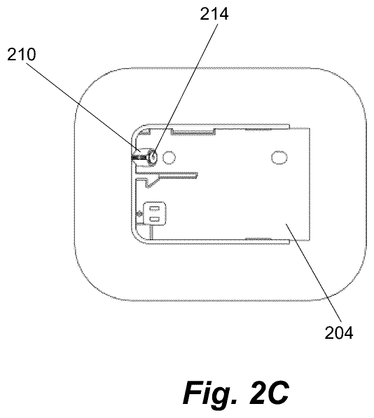

FIGS. 2A-2C depict an infusion pump system 200 according to another embodiment of the disclosure. System 200 can include an infusion pump 202 configured as a patch pump that is worn directly on the body of the user with an adhesive patch 206. Pump 202 can be affixed to the body of the user with an adhesive patch 206 carrying a tray 204 thereon that releasably attaches the pump 202 to the tray 204. Pump 202 can be configured to deliver medicament to the user through a cannula 208 extending directly beneath the pump 202 and adhesive patch 206. Referring to FIG. 2C, in embodiments tray 204 can include a cannula port 210 through which cannula 208 is inserted. The cannula port 210 can contain a septum 214 therein that facilitates transfer of insulin from the pump 202 to the cannula 208.

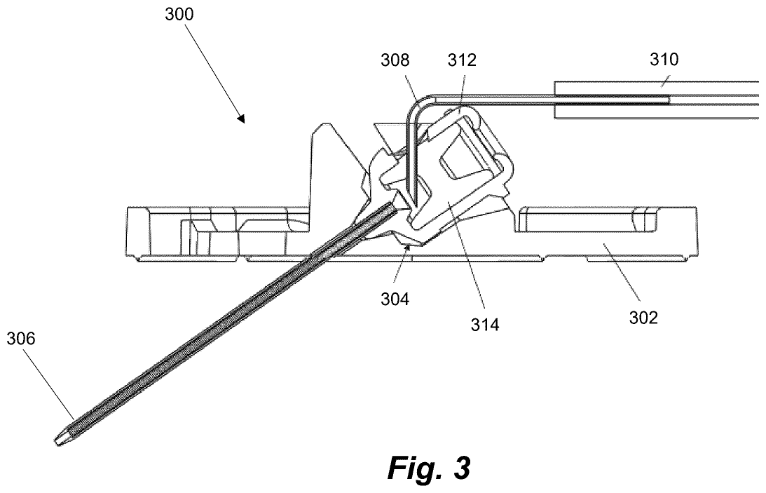

FIG. 3 depicts an infusion hub 300 of an infusion set according to an embodiment of the disclosure. Hub 300 can include a patch 302 or other mechanism configured to adhere to the patient and a barrel 304 connected to a cannula 306. A fluid introducer needle 308 can interface tubing 310 with the hub 300. The barrel 304 can include a mechanical housing 312 configured to house a septum 314. The fluid introducer needle 308 can be configured to pierce the septum 314 to deliver fluid from the tubing 310 to the cannula 306. The fluid path for the insulin medication, therefore, can run from a reservoir of a pump through infusion tubing extending from the pump, through one or more connectors connecting the infusion tubing of the pump with tubing 310 of an infusion set, through the tubing 310 of the infusion set, to the barrel 304 and to the patient via the cannula 306. Although infusion hub 300 is depicted as being employed with a length of tubing 310 that may interface with tubing extending from a pump such as depicted in FIGS. 1A-1B, the components of infusion hub 300 such as the septum 314, cannula 306, etc. can be adapted to be incorporated into a patch pump system such as the system depicted in FIGS. 2A-2B.

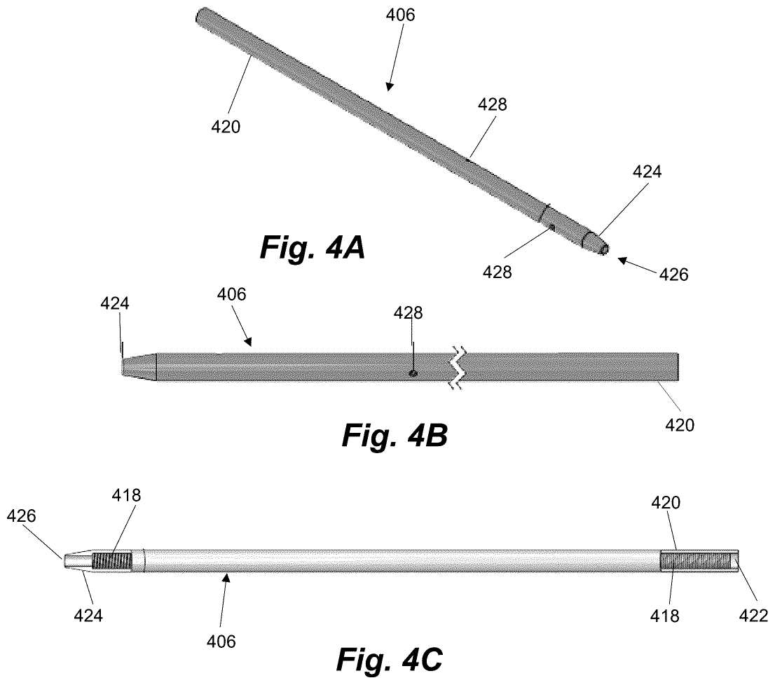

FIGS. 4A-4C depict a cannula 406 for use in an infusion set, with a patch pump, or to otherwise deliver a liquid medicament into a user according to an aspect of the disclosure. Cannula 406 includes a tube wall 420 having an inner lumen 422 through which medicament can be delivered to a user. A coil 418 can be disposed within inner lumen 422 of cannula 406. Tube wall 420 may comprise a softer, more flexible polymer material, such as, for example, a thermoplastic elastomer and coil 422 can comprise a harder, more rigid material such as, for example, stainless steel. Cannula 406 can further include a tapered distal end 424 having a distal opening 426 and one or more infusion holes 428 along the tube wall.

As noted above, providing a coil inside of a cannula has been found to provide a number of benefits. In some examples, the coil is comprised of a stainless-steel material that is co-extruded to have a polymer sleeve over its external surface. While cannulas with internal coils provide significant advantages, there are technical challenges and increased costs associated with the manufacturing process. For example, the coils must be manually inserted on a long mandrel, the coils must be manually spaced and aligned along the length of the mandrel, care must be taken so that the coils do not shift during the extrusion process, the mandrel must be removed from the extruded coils, and the extruded coils must be cut to size. These requirements, alone or in combination with one another, can be a challenge for large scale manufacturing.

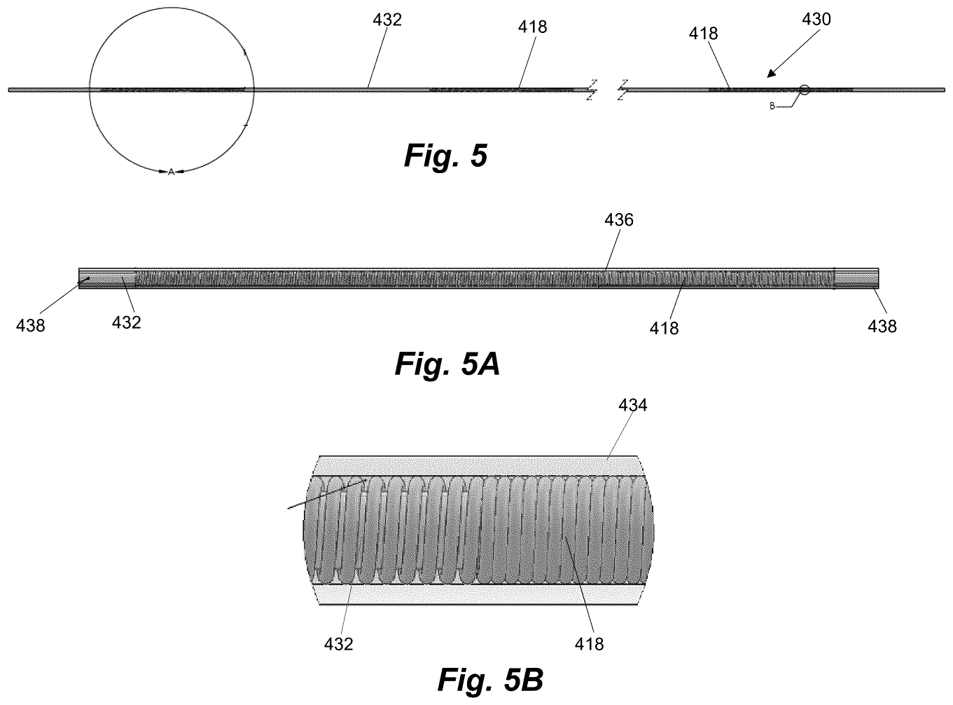

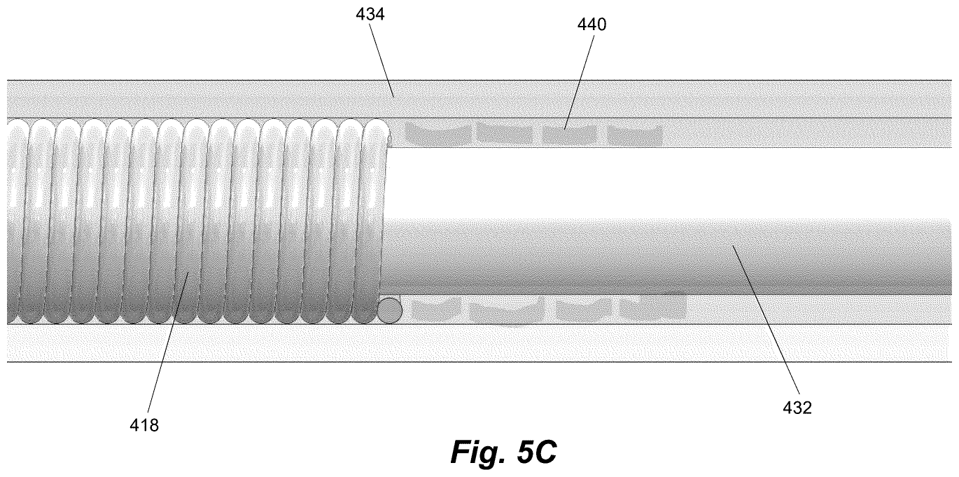

To manufacture cannulas with an internal coil as described above, a plurality of coils can be arranged on a mandrel that may be comprised of, for example, stainless steel. The mandrel and coils are then fed through an extrusion machine that provides a polymer (e.g., a thermoplastic elastomer) overcoat around the coils and mandrel. FIGS. 5A-5C depict an assembly 430 at this stage of the process. A plurality of coils 418 are positioned on a mandrel 432 in a spaced apart fashion, such as, for example, 1 inch apart. A tubular wall 434 provided in the extruder surrounds the coils 418 and mandrel 432. The assembly 430 would then be processed into a plurality (e.g., 10) sections 436 by cutting the assembly between the coils (the mandrel can be removed before or after cutting the sections). As depicted in FIG. 5A, each section 436 includes a length of tubing 438 on each end that extends beyond the coil 418. Each cut section 436 can be processed into a cannula by providing the tapered distal end 424 and infusion holes 428 (See FIGS. 4A-4C). Referring to FIG. 5C, there is an open space 440 between each coil on the mandrel 432. It has been found that it is difficult to maintain the spacing of the coils 418 when assembling the coils 418 on the mandrel 432 and that the forces applied by the extrusion machine can also cause the coils 418 to move on the mandrel 432. This can result in coils 418 that are too close together within the tubular wall 434 of the assembly 430 to provide a sufficient length of tubing 438 extending beyond the coils 418 for formation of the tapered distal end 434 of the cannula.

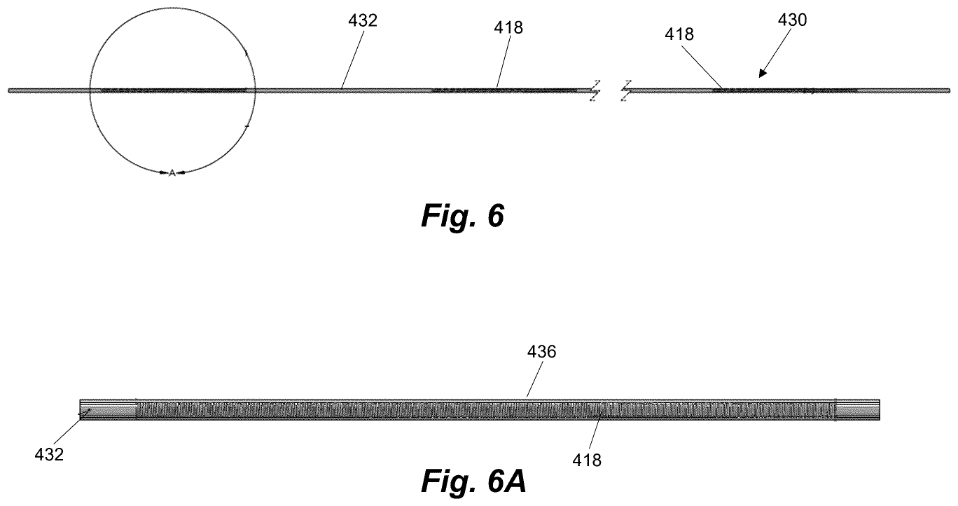

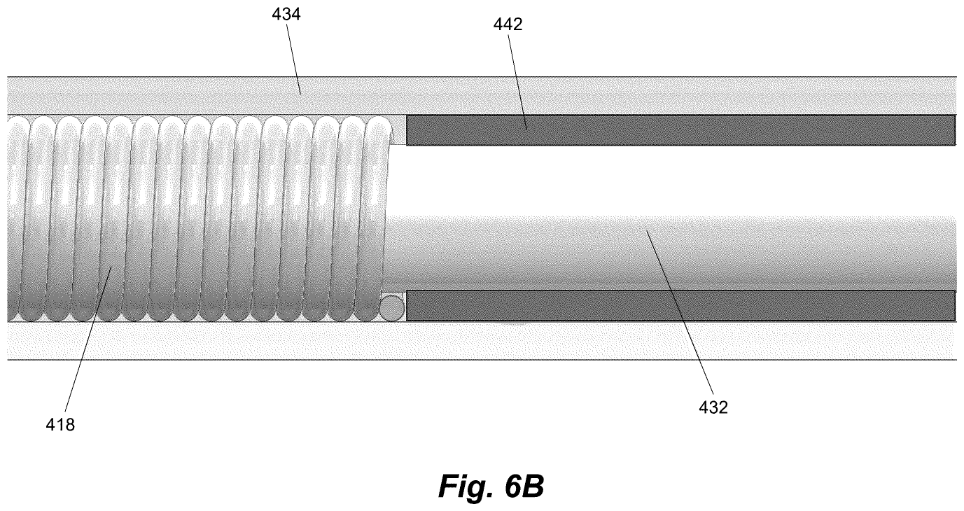

Referring now to FIGS. 6-6B, the present disclosure solves the above issue by providing spacers 442 along the portions of mandrel 432 between the coils 418. Spacers 442 can be tubular members inserted over the mandrel between the coils 418 having the same inner diameter and outer diameter as the coils 418. In embodiments, spacers 442 are comprised of a polymer material, such as a thermoplastic elastomer. Spacers 442 will prevent shifting of the coils 418 both during assembly on the mandrel 432 and during the extrusion process, which will ensure sufficient material extending beyond the coil for creation of the tapered distal end for the cannula. Use of spacers 442 having a matching outer diameter to the coils 418 also provides a more uniform outer wall 434 as the tube wall 420 extends from areas having coils 418 to areas not having coils (i.e. areas having spacers 442). The spacer 442 can be removed from the cut section 436 prior to formation of the tip.

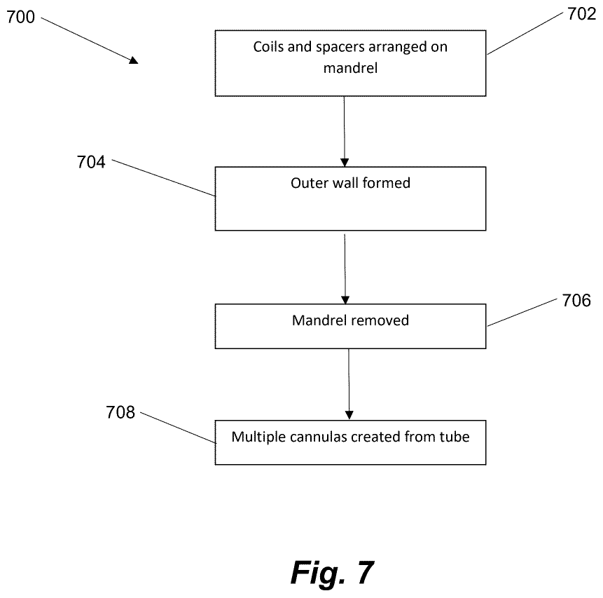

Referring now to FIG. 7, a flowchart of method steps 700 for a method of manufacturing a plurality of cannulas such as, for example, cannula 406, for use in an infusion set, patch pump, or other infusion mechanism is depicted. At step 702, a plurality of coils and a plurality of spacers are arranged on a mandrel as described above in alternating fashion. This assembly is processed through an extrusion device at step 504 to form the outer tubular wall. Following extrusion, the mandrel can be removed at step 506 leaving an elongate tube having alternating coil and spacer sections. The tube can be processed into multiple cannulas at step 708. This can include, for example, cutting the tube to length, removing the remaining spacer material, providing a tapered tip at the distal end and laser drilling infusion holes in the sides of the tube. The cannulas can then be further processed for the particular infusion mechanism with which the cannula would be used such as an infusion set, patch pump, etc.

In an embodiment, a method of manufacturing a plurality of cannulas configured to facilitate delivery of medicament to a user can including arranging a plurality of coils on a mandrel and providing a spacer on the mandrel between each of the coils to form an assembly having alternating coils and spacers along the mandrel. The assembly can be processed through an extrusion device to form an outer tube around the assembly. The processed assembly can be cut adjacent both ends of each coil to create a plurality of cannula sections. A cannula configured to facilitate delivery of medicament can be formed from each cannula section.

In some embodiments, cutting the processed mandrel assembly adjacent both ends of each coil to create a plurality of cannula sections includes providing each cannula section with a length of tubing extending beyond the coil on at least one end of the cannula section.

In some embodiments, forming the cannula includes forming a tapered distal end for the cannula at the length of tubing extending beyond the coil on one end of the cannula section.

In some embodiments, the method further comprises removing a portion of a spacer from the length of tubing prior to forming the tapered distal end.

In some embodiments, forming the cannula includes forming one or more infusion holes in a side of the cannula section.

In some embodiments, each spacer has a same inner diameter and outer diameter as each of the plurality of coils.

In some embodiments, each spacer is comprised of a polymer material.

In some embodiments, the method further comprises removing the mandrel prior to cutting the assembly.

In some embodiments, the method further comprises attaching a barrel to a proximal end of each cannula to provide a cannula assembly.

In some embodiments, the outer tube comprises a polymer and the coils comprise a metal.

In an embodiment, a cannula configured to facilitate delivery of medicament to a user is formed by a process comprising arranging a plurality of coils on a mandrel and providing a spacer on the mandrel between each of the coils to form an assembly having alternating coils and spacers along the mandrel. The assembly can be processed through an extrusion device to form an outer tube around the assembly. The processed assembly can be cut adjacent both ends of each coil to create a plurality of cannula sections. A cannula configured to facilitate delivery of medicament can be formed from each cannula section.

In some embodiments, cutting the processed mandrel assembly adjacent both ends of each coil to create a plurality of cannula sections in the process includes providing each cannula section with a length of tubing extending beyond the coil on at least one end of the cannula section.

In some embodiments, forming the cannula with the process includes forming a tapered distal end for the cannula at the length of tubing extending beyond the coil on one end of the cannula section.

In some embodiments, the process further comprises removing a portion of a spacer from the length of tubing prior to forming the tapered distal end.

In some embodiments, forming the cannula with the process includes forming one or more infusion holes in a side of the cannula section.

In some embodiments, each spacer used in the process has a same inner diameter and outer diameter as each of the plurality of coils.

In some embodiments, each spacer used in the process is comprised of a polymer material.

In some embodiments, the process further comprises removing the mandrel prior to cutting the assembly.

In some embodiments, the process further comprises attaching a barrel to a proximal end of each cannula to provide a cannula assembly.

In some embodiments, the outer tube comprises a polymer and the coils comprise a metal.

Although the infusion pump embodiments herein are specifically described primarily with respect to the delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425; and 12,214,159 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924; 2024/0399051; 2024/408303; 2024/0416032; 2024/0416033; 2025/0099674; 2025/0099675 2025/0099678; 2025/0099679; and 2025/0108162 and commonly owned U.S. patent application Ser. Nos. 17/368,968; 17/896,492; 18/398,543; 18/962,169; 19/003,140; 19/003,164 and 19/119,554.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

The invention claimed is:

1. A method of manufacturing a plurality of cannulas configured to facilitate delivery of medicament to a user, comprising:
arranging a plurality of coils on a mandrel;
providing a spacer on the mandrel between each of the coils to form an assembly having alternating coils and spacers along the mandrel;
processing the assembly through an extrusion device to form an outer tube around the assembly;
cutting the processed assembly adjacent both ends of each coil to create a plurality of cannula sections; and
forming a cannula configured to facilitate delivery of medicament from each cannula section.

2. The method of claim 1, wherein cutting the processed mandrel assembly adjacent both ends of each coil to create a plurality of cannula sections includes providing each cannula section with a length of tubing extending beyond the coil on at least one end of the cannula section.

3. The method of claim 2, wherein forming the cannula includes forming a tapered distal end for the cannula at the length of tubing extending beyond the coil on one end of the cannula section.

4. The method of claim 3, further comprising removing a portion of a spacer from the length of tubing prior to forming the tapered distal end.

5. The method of claim 1, wherein forming the cannula includes forming one or more infusion holes in a side of the cannula section.

6. The method of claim 1, wherein each spacer has a same inner diameter and outer diameter as each of the plurality of coils.

7. The method of claim 1, wherein each spacer is comprised of a polymer material.

8. The method of claim 1, further comprising removing the mandrel prior to cutting the assembly.

9. The method of claim 1, further comprising attaching a barrel to a proximal end of each cannula to provide a cannula assembly.

10. The method of claim 1, wherein the outer tube comprises a polymer and the coils comprise a metal.

11. A cannula configured to facilitate delivery of medicament to a user formed by a process comprising:
arranging a plurality of coils on a mandrel;
providing a spacer on the mandrel between each of the coils to form an assembly having alternating coils and spacers along the mandrel;
processing the assembly through an extrusion device to form an outer tube around the assembly;
cutting the processed assembly adjacent both ends of each coil to create a plurality of cannula sections; and
forming a cannula configured to facilitate delivery of medicament from each cannula section.

12. The cannula of claim 11, wherein cutting the processed mandrel assembly adjacent both ends of each coil to create a plurality of cannula sections in the process includes providing each cannula section with a length of tubing extending beyond the coil on at least one end of the cannula section.

13. The cannula of claim 12, wherein forming the cannula with the process includes forming a tapered distal end for the cannula at the length of tubing extending beyond the coil on one end of the cannula section.

14. The cannula of claim 13, wherein the process further comprises removing a portion of a spacer from the length of tubing prior to forming the tapered distal end.

15. The cannula of claim 11, wherein forming the cannula with the process includes forming one or more infusion holes in a side of the cannula section.

16. The cannula of claim 11, wherein each spacer used in the process has a same inner diameter and outer diameter as each of the plurality of coils.

17. The cannula of claim 11, wherein each spacer used in the process is comprised of a polymer material.

18. The cannula of claim 11, wherein the process further comprises removing the mandrel prior to cutting the assembly.

19. The cannula of claim 11, wherein the process further comprises attaching a barrel to a proximal end of each cannula to provide a cannula assembly.

20. The cannula of claim 11, wherein the outer tube comprises a polymer and the coils comprise a metal.
